(19) United States
(12) Patent Application Publication
(10) Pub. No.: US 2010/0173921 A1
(43) Pub. Date: Jul. 8, 2010

(54) Antiretroviral Solid Oral Composition

(75) Inventors: Amar Lulla, Maharashtra (IN); Geena Malhotra, Maharashtra (IN)

Correspondence Address: CONLEY ROSE, P.C., 5601 GRANITE PARKWAY, SUITE 750, PLANO, TX, 75024, US

(73) Assignee: CIPLA LIMITED, Mumbai Central (IN)

(21) Appl. No.: 12/377,038

(22) PCT Filed: Aug. 10, 2007
(86) PCT No.: PCT/GB2007/003061

§ 371 Date: Apr. 15, 2009

(30) Foreign Application Priority Data
Aug. 10, 2006 (IN) 1269/MUM/2006
Jun. 27, 2007 (IN) 1227/MUM/2007

(51) Int. Cl. A61K 31/513; A61P 31/18

(52) U.S. Cl. 514/274

(57) ABSTRACT

The present invention provides an antiretroviral solid oral composition comprising one or more antiretroviral drugs, for example protease inhibitors such as lopinavir, ritonavir or a combination thereof with one or more excipients. The invention provides a composition which is smaller for a given amount of said active substance and possesses taste masking property and a process for preparing the composition. The present invention also provides an antiretroviral solid oral composition comprising one or more antiretroviral drugs, for example protease inhibitors such as lopinavir, ritonavir or a combination thereof with at least one water insoluble polymer, wherein the ratio of drug to polymer in the composition ranges from about 1:1 to about 1:6, and a process for preparing the composition.

### FIELD OF INVENTION

The present invention relates to antiretroviral solid oral compositions and a process for their manufacture.

### BACKGROUND AND PRIOR ART

Acquired Immune Deficiency Syndrome (AIDS) causes a gradual breakdown of the body's immune system as well as progressive deterioration of the central and peripheral nervous systems. Two distinct retroviruses, human immunodeficiency virus (HIV) type-1 (HIV-I) or type-2 (HIV-2), have been etiologically linked to the immunosuppressive disease, acquired immunodeficiency syndrome (AIDS). HIV seropositive individuals are initially asymptomatic but typically develop AIDS related complex (ARC) followed by AIDS. Affected individuals exhibit severe immunosuppression, which predisposes them to debilitating and ultimately fatal opportunistic infections. Retroviral replication routinely features post-translational processing of polyproteins. This processing is accomplished by virally encoded HIV protease enzyme. This yields mature polypeptides that will subsequently aid in the formation and function of infectious virus. If this molecular processing is stifled, then the normal production of HIV is terminated. Therefore, inhibitors of HIV protease may function as anti-HIV viral agents.

There are various compositions comprising HIV protease inhibitors and methods of preparing the same.

Ritonavir and its salts are first described in U.S. Pat. No. 5,541,206. The said patent describes the structure of Ritonavir and the processes for its preparation. Further it describes pharmaceutical compositions and process for making compositions comprising Ritonavir. The compositions described are administered orally, parenterally, sublingually, by inhalation spray, rectally, or topically in dosage unit formulations containing conventional nontoxic pharmaceutically acceptable carriers, adjuvants, and vehicles. Solid dosage forms for oral administration include capsules, tablets, pills, powders, and granules.

Lopinavir and its salts are first described in U.S. Pat. No. 5,914,332. The said patent describes the structure of Lopinavir and the processes for its preparation. Further it describes pharmaceutical compositions comprising lopinavir. The patent further describes a preferred dosage form as a soft elastic gelatin capsule (SEC) or a hard gelatin capsule. The combination of Lopinavir with Ritonavir and the use for inhibition or treatment of HIV or AIDS in combination is also described in the said patent. Ritonavir on co-administration with lopinavir causes an improvement in the pharmacokinetics (i.e., increases half-life, increases the time to peak plasma concentration, increases blood levels) of lopinavir.

WO9822106 describes a liquid pharmaceutical composition of compounds which are inhibitors of HIV protease with improved oral bioavailability. This application in particular, describes a composition in the form of a solution which comprises (a) the HIV protease inhibitor, (b) a pharmaceutically acceptable organic solvent and, optionally, (c) a surfactant. It is further described that the composition can be optionally encapsulated in either hard gelatin capsules or soft elastic capsules (SEC). The preferred HIV protease inhibitor is a Lopinavir/Ritonavir combination. The above process involves a complex manufacturing process.

WO02096395 relates to soft elastic capsules and HIV protease inhibiting compounds contained in the soft elastic capsule. The application describes soft elastic capsules that have a fill, which includes pharmaceutical agents, an alcohol, and fatty acid; and a shell, which includes gelatin and plasticizing agents. It is well known in the art that there is a limited choice of excipients/carriers compatible with gelatin. In general capsules have crosslinking problems and to overcome these problems, fillers and stabilizers like citric acid, glycine has to be incorporated.

It is further well known in the art that pharmaceutical compositions in solid dosage form have great stability, less risk of chemical interaction between different medicaments, smaller bulk, accurate dosage, and ease of production. However, geriatric and pediatric patients experience difficulty in swallowing larger sized tablets wherein large size results in esophageal damage due to physical characteristics of the dosage form if it is not swallowed properly, which leads to poor patient compliance. Apart from above, palatability and medicament acceptance are also one of the most important parameters governing patient compliance. Oral administration of bitter drugs with an acceptable degree of palatability is a key issue for health care providers, especially for pediatric patients.

Tablets comprising one or more antiretroviral drugs are known, but involve a melt extrusion process in order to be prepared. Melt extrusion generally requires a large number of excipients to be used during processing, so will result in a large tablet. Typically, tablets prepared using a melt extrusion process weigh from about 1200 to about 1300 mg.

Hence, there still remains a need to formulate a solid oral dosage form with a minimal weight and taste masking property with enhanced bioavailability.

The present inventors have surprisingly found that a solid oral composition with a minimal weight comprising suitable excipients within limited ranges may be achieved by the use of a simple manufacturing process. The composition has enhanced bioavailability and increased palatability due to a taste masking property.

### OBJECT OF THE INVENTION

The object of the present invention is to provide a solid oral composition weighing a minimal amount.

Another object of the present invention is to provide a solid oral composition with a taste masking property and better patient compliance.

Still another object of the present invention is to provide a solid oral composition which is easy to manufacture.

### SUMMARY OF THE INVENTION

According to a first aspect of the present invention, there is provided a solid oral composition comprising one or more antiretroviral drugs, or their pharmaceutically acceptable salts, solvates or hydrates and at least one water insoluble polymer, wherein the ratio of drug to polymer in the composition ranges from about 1:1 to about 1:6. In an embodiment, said composition possesses a taste masking property. In another embodiment, the or each antiretroviral drug is selected from protease inhibitors or their salts, solvates or hydrates. Suitably, the composition comprises two protease inhibitors or their pharmaceutically acceptable salts, solvates or hydrates. Preferably, the or each protease inhibitor is selected from lopinavir, ritonavir, amprenavir, saquinavir or their pharmaceutically acceptable salts, solvates or hydrates. More preferably, the two protease inhibitors are lopinavir and ritonavir or their pharmaceutically acceptable salts, solvates or hydrates. It will be appreciated that the drugs may also be in the form of their enantiomers, derivatives, polymorphs or prodrugs. Most preferably, the two protease inhibitors are lopinavir and ritonavir.

In an embodiment, the or each water insoluble polymer is selected from the group consisting of acrylic copolymers; polyvinylacetate; and cellulose derivatives. Suitably, the or each acrylic copolymer is selected from Eudragit E100, Eudragit EPO, Eudragit L30D-55, Eudragit FS30D, Eudragit RL30D, Eudragit RS30D, Eudragit NE30D and Acryl-Eze. Preferably, the water insoluble polymer is Eudragit E100. Optionally, the polyvinylacetate comprises Kollicoat SR 3OD. Suitably, the or each cellulose derivative is selected from the group consisting of ethylcellulose and a cellulose acetate. Optionally, the or each cellulose acetate is selected from Surelease, Aquacoat ECD and Aquacoat CPD.

In another embodiment, the composition further comprises at least one water soluble polymer. Typically, the water soluble polymer is selected from the group of homopolymers and co-polymers of N-vinyl lactams, co-polymers of polyvinylpyrrolidone and vinyl acetate, co-polymers of N-vinyl pyrrolidone and vinyl acetate or vinyl propionate; high molecular polyalkylene oxides and co-polymers of ethylene oxide and propylene oxide. The water soluble polymer may be a homopolymer or co-polymer of N-vinyl pyrrolidone. The homopolymer of N-vinyl pyrrolidone may be polyvinylpyrrolidone. Alternatively, the water soluble polymer is a copolymer of polyvinyl pyrrolidone and vinyl acetate, preferably Kollidon VA 64. Suitably, the high molecular polyalkylene oxides are selected from the group consisting of polyethylene oxide and polypropylene oxide.

In a further embodiment, the composition further comprises a plasticizer. Optionally, the plasticizer is selected from the group consisting of a polysorbate, a citrate ester, propylene glycol, glycerin, low molecular weight polyethylene glycol, triacetin, dibutyl sebacate, tributyl sebacate, dibutyltartrate and dibutyl phthalate. The polysorbate may be selected from the group consisting of sorbitan monolaurate, sorbitan monopalmitate, sorbitan monostearate and sorbitan monoisostearate. The citrate ester may be triethyl citrate or citrate phthalate. Typically, the plasticizer is present in an amount of up to about 10% of the weight of polymer.

In a further embodiment, the composition further comprises one or more bulking agents. Typically, the or each bulking agents are selected from the group consisting of saccharides, sugar alcohols, powdered cellulose, microcrystalline cellulose, purified sugar and derivatives thereof. The saccharides may be selected from the group consisting of monosaccharides, disaccharides or polysaccharides. The sugar alcohols may be selected from the group consisting of arabinose, lactose, dextrose, sucrose, fructose, maltose, mannitol, erythritol, sorbitol, xylitol or lactitol. Preferably, the bulking agent comprises purified sugar.

In another embodiment, the composition further comprises one or more flavourants. Suitably, the or each flavourant is selected from citric acid, tartaric acid, lactic acid, and other natural flavourants.

The compositions may be in the form of granules, pellets or tablets. The administration of granulate form or pellet form can be by filling in sachets which are suitable for ingestion.

According to a second aspect of the present invention, there is provided a process for preparing a solid oral composition comprising one or more antiretroviral drugs, or their pharmaceutically acceptable salts, solvates or hydrates, at least one water insoluble polymer and one or more pharmaceutically acceptable excipients, the process comprising melt extruding comprising the steps: (a) preparing a homogeneous melt of the or each drug; the or each water insoluble polymer and the or each excipients; (b) cooling the melt obtained in step (a); (c) allowing the cooled melt to solidify to obtain extrudates; and (d) processing the extrudates into a desired shape. Optionally, step (a) is carried out at a temperature ranging from about 70° C. to about 200° C. typically out at a temperature ranging from about 90° C. to about 150° C. It will be appreciated that the or drugs may be in the form of their pharmaceutically acceptable enantiomers, derivatives, polymorphs or prodrugs.

In an embodiment, step (d) comprises shaping the extrudates into a tablet. Alternatively, step (d) comprises cutting the extrudate into pieces and can further processing the cut extrudates into suitable dosage forms. Alternatively, step (d) comprises milling and grinding the extrudates to form granules.

The process may involve preparing a composition as defined in the first aspect above. According to a third aspect of the present invention, there is provided the use of at least one water insoluble polymer in the preparation of a solid oral composition comprising one or more antiretroviral drugs, or their pharmaceutically acceptable salts, solvates or hydrates, wherein the ratio of drug to polymer in the composition ranges from about 1:1 to about 1:6. The composition may be as described above in the first aspect.

According to a fourth aspect of the present invention, there is provided a composition prepared according to the process described in the second aspect above.

According to a fifth aspect of the present invention, there is provided a process for preparing a solid oral composition comprising one or more antiretroviral drugs or their pharmaceutically acceptable salts, solvates or hydrates, the process comprising: (a) melt granulating one or more solubility enhancers and one or more first pharmaceutically acceptable excipients with the or each drugs in purified water to form a granulated material; (b) sieving the granulated material; (c) drying the sieved granulated material to form dried granules; (d) lubricating the dried granules with one or more lubricants and one or more second pharmaceutically acceptable excipients; and (e) optionally further processing the lubricated dried granules. Suitably, the composition comprises two antiretroviral drugs or their pharmaceutically acceptable salts, solvates or hydrates. In an embodiment, the or each antiretroviral drug is a protease inhibitor selected from the group consisting of lopinavir, ritonavir, amprenavir and saquinavir or a pharmaceutically acceptable salt, solvate, or hydrate thereof. Preferably, the or each protease inhibitor is selected from lopinavir and ritonavir or their pharmaceutically acceptable salts, solvates, or hydrates. More preferably, the composition is a combination of lopinavir and ritonavir or their pharmaceutically acceptable salts, pharmaceutically acceptable solvates or hydrates. It will be appreciated that the drugs may be in the form of their enantiomers, derivatives, polymorphs or prodrugs. Still more preferably, the composition is a combination of lopinavir and ritonavir.

In an embodiment, the solid oral composition is a tablet and step (e) comprises compressing the lubricated dried granules. The process may further comprise: (f) seal coating the tablet. Alternatively, the process further comprise: (f) film coating the tablet. Alternatively, the process may further comprise: (f) seal coating the tablet; and (g) film coating the seal coated tablet. In an embodiment, wherein the seal coat material is a hydroxypropylmethylcellulose. Typically, the hydroxypropylmethylcellulose is selected from hydroxypropyl methylcellulose (HPMC) 6CPS to hydroxypropyl methylcellulose (HPMC) 15CPS.

In an embodiment, the solid oral composition is a capsule and step (e) comprises filling capsules with the lubricated dried granules.

In an embodiment, the solid oral composition is in the form of granules suitable for direct administration and there is no step (e).

In an embodiment, the or each solubility enhancers are selected from the group consisting of stearoyl macrogol glyceride, a polysorbate, and polyoxyl castor oil. Typically, the solubility enhancer is stearoyl macrogol glyceride. Optionally, the polysorbate comprises sorbitan monolaurate (Span 20).

In an embodiment, the first pharmaceutically acceptable excipients and second pharmaceutically acceptable excipients independently of one another are selected from the group consisting of polymers, fillers or diluents, surfactants, solubility enhancers, disintegrants, binders, lubricants, non-ionic solubilisers, glidants and combinations thereof.

In an embodiment, the or each binder is selected from the group consisting of copovidone, celluloses, polyvinyl pyrrolidone, starches and other pharmaceutically acceptable substances with cohesive properties. The cellulose may be selected from the group consisting of hydroxypropyl methylcellulose, hydroxypropyl cellulose and microcrystalline cellulose.

In an embodiment, the first pharmaceutically acceptable excipients and second pharmaceutically acceptable excipients independently of one another comprise one or more diluents and one or more disintegrants.

In an embodiment, the or each diluents are selected from the group consisting of calcium silicate, pregelatinized starch, croscarmellose sodium, sodium starch glycollate and microcrystalline cellulose. Suitably, the diluent is microcrystalline cellulose and is present in an amount of about 10 mg to about 300 mg. Alternatively, the diluent is calcium silicate and is present in an amount of about 100 mg to about 300 mg.

In an embodiment, the disintegrant is present in an amount of about 50 mg to about 250 mg.

In an embodiment, wherein the solubility enhancer is present in an amount of about 10 mg to about 100 mg.

In an embodiment, the or each disintegrants are selected from the group consisting of crospovidone, ac-di-sol and sodium starch glycollate.

In an embodiment, the or each lubricants are selected from the group consisting of stearic acid, its derivatives or esters, colloidal silicon dioxide and talc. Suitably, the or each lubricants comprise an ester of stearic acid. Preferably, the lubricant comprises magnesium stearate or calcium stearate.

In an embodiment, the non-ionic solubiliser comprises chremophore.

In an embodiment, the composition has a taste-masking property.

In an embodiment, the composition is smaller for a given amount of the or each drugs.

According to a further aspect of the present invention, there is provided a process for preparing a solid oral composition comprising one or more antiretroviral drugs or their pharmaceutically acceptable salts, solvates or hydrates, the composition having a taste-masking property, the process comprising: (a) melt granulating one or more solubility enhancers and one or more first pharmaceutically acceptable excipients with the or each drugs in purified water to form a granulated material; (b) sieving the granulated material; (c) drying the sieved granulated material to form dried granules; (d) lubricating the dried granules with one or more lubricants and one or more second pharmaceutically acceptable excipients; and (e) optionally further processing the lubricated dried granules. It will be appreciated that the or each drugs may be in the form of their pharmaceutically acceptable enantiomers, derivatives, polymorphs or prodrugs.

According to yet another aspect of the present invention, there is provided a solid oral composition prepared according to the process described in the fifth aspect above.

Thus, the present invention provides a solid oral composition, suitably a tablet, comprising one or more antiretroviral drugs or their pharmaceutically acceptable salts, solvates or hydrates, and one or more pharmaceutically acceptable excipients, wherein the composition has been prepared by melt granulation. The composition is smaller for a given amount of said active substance. Typically, a tablet according to the present invention will not weigh more than about 1050 mg. The composition may comprise two antiretroviral drugs or their pharmaceutically acceptable salts, solvates or hydrates. In an embodiment, the or each antiretroviral drug is a protease inhibitor selected from the consisting of lopinavir, ritonavir, amprenavir and saquinavir or a pharmaceutically acceptable salt, solvate or hydrate thereof. Suitably, the or each antiretroviral drug is selected from lopinavir and ritonavir or their pharmaceutically acceptable salts, solvates or hydrates Preferably, the composition is a combination of lopinavir and ritonavir or their pharmaceutically acceptable salts, pharmaceutically acceptable solvates or hydrates. It will be appreciated that the or each drugs may be in the form of their enantiomers, derivatives, polymorphs or prodrugs. More preferably, the composition is a combination of lopinavir and ritonavir.

In an embodiment, the composition comprises one or more excipients comprising one or more polymers, fillers or diluents, surfactants, solubility enhancers, disintegrants, binders, lubricants, non-ionic solubilisers and glidants or combinations thereof.

The or each diluent may be selected from calcium silicate, microcrystalline cellulose, pregelatinized starch, croscarmellose sodium or sodium starch glycollate.

The or each disintegrant may be selected from the group consisting of crospovidone, ac-di-sol or sodium starch glycollate.

The or each binder may be selected from the group consisting of copovidone, celluloses such as hydroxypropyl methylcellulose, hydroxypropyl cellulose, microcrystalline cellulose, polyvinyl pyrrolidone, starches and other pharmaceutically acceptable substances with cohesive properties.

The or each solubility enhancer may be selected from stearoyl macrogol glyceride, a polysorbate or polyoxyl castor oil. Suitably, the polysorbate is sorbitan monolaurate (Span 20)

The or each lubricant may be selected from the group consisting of stearic acid, its derivatives or esters, talc or silicon dioxide. Optionally, the or each lubricant is an ester of stearic acid. Suitably, the lubricant is colloidal silicon dioxide. Preferably, the lubricant is magnesium stearate or calcium stearate.

In an embodiment the composition further comprises a film coating.

In an alternative embodiment, the composition further comprises a seal coat material. The seal coat material may be a hydroxypropylmethylcellulose. For example, the hydroxypropylmethylcellulose may be selected from hydroxypropyl methylcellulose (HPMC) 6CPS to hydroxypropyl methylcellulose (HPMC) 15CPS. The seal coated composition may further comprise a film coating.

In an embodiment, the diluent is calcium silicate and is present in an amount of about 100 mg to about 300 mg. Suitably, the calcium silicate and is present in an amount of about 180 mg to about 220 mg.

In an embodiment, the diluent is microcrystalline cellulose and is present in an amount of about 10 mg to about 300 mg. Suitably, the microcrystalline cellulose is present in an amount of about 30 mg to about 60 mg.

In an embodiment, the disintegrant is present in an amount of about 50 mg to about 250 mg. Suitably, the disintegrant is present in an amount of about 100 mg to about 200 mg.

In an embodiment, the solubility enhancer is present in an amount of about 10 mg to about 100 mg. Suitably, the solubility enhancer is present in an amount of about 40 mg to about 60 mg.

In another embodiment, the composition possesses a taste masking property.

### DETAILED DESCRIPTION OF THE INVENTION

As discussed above, the present invention relates to a solid oral composition comprising one or more antiretroviral drugs and one or more pharmaceutically acceptable excipients wherein the composition is smaller for a given amount of said active substance. The most preferable antiretroviral drugs to be used are protease inhibitors such as lopinavir, ritonavir, amprenavir, saquinavir and others or their corresponding pharmaceutically acceptable salts, solvates, or hydrates. The or each protease inhibitor may also be a pharmaceutically acceptable enantiomer, derivative, polymorph or prodrug thereof.

The said composition comprises one or more excipients which include, but are not limited to polymers, fillers or diluents, surfactants, bioavailability enhancer, disintegrants, binders, lubricants, non-ionic solubilisers, glidants and others, and combinations thereof.

According to a preferred embodiment, the diluent of the present invention in the dry mix is selected from the group consisting of calcium silicate, pregelatinized starch, croscarmellose sodium or sodium starch glycollate. Pregelatinized starch can be present in the range of 50-250 mg, croscarmellose sodium in the range of 50-100 mg and sodium starch glycollate in the range of 50-100 mg, preferably the diluent used is calcium silicate and it may be present in the range of 100-300 mg; the most preferred range being 180-220 mg.

The diluent of the present invention in blending may be microcrystalline cellulose. It may be present in the range of 10-300 mg; the most preferred range being 30-60 mg.

The binder can be selected from the group consisting of copovidone, celluloses such as hydroxypropyl methylcellulose, hydroxypropyl cellulose, microcrystalline cellulose, polyvinyl pyrrolidone, starches and other pharmaceutically acceptable substances with cohesive properties.

The disintegrants can be selected from the group consisting of crospovidone, ac-di-sol and sodium starch glycollate. They may be present in the range of 50-250 mg; the most preferred range being 100-200 mg.

The solubility enhancer can be selected from stearoyl macrogol glyceride, sorbitan monolaurate (Span 20), Polyoxyl castor oil & more preferably stearoyl macrogol glyceride. It may be present in the range of 10-100 mg; the most preferred range being 40-60 mg.

The lubricants can be selected from the group consisting of stearic acid and its derivatives or esters like magnesium stearate and calcium stearate, stearyl fumarate; colloidal silicone dioxide; talc.

The seal coating ingredient can be selected from HPMC 6 CPS, or HPMC 6 CPS to HPMC 15CPS grade. The HPMC component of the seal coating may be mixed with solvents such as methylene chloride and isopropyl alcohol or mixtures thereof. The seal coating may also comprise talc.

The composition can be further film coated with Ready colour mix system.

This was a surprising finding that due to the incorporation of actives i.e. at least one protease inhibitor such as lopinavir or ritonavir or a combination of lopinavir and ritonavir into the solubility enhancer such as stearoyl macrogol glyceride, there resulted in an increase in the dissolution rate of the drugs, ultimately leading to improved drug bioavailability due to an interaction between the drug and the excipient.

The present invention can be administered orally through the known solid dosage forms including tablet, capsule (filled with granules or pellets) or individually granules or pellets can be administered directly. The capsules may be hard gelatin capsules. Sachets may be filled with the granules or pellets that are suitable for direct administration.

The present invention can be manufactured through various techniques or processes including melt granulation, melt extrusion, spray drying and solution evaporation.

According to a preferred embodiment, the present invention may be in the form of tablet processed by melt granulation technique. One preferred embodiment is as follows. Stearoyl macrogol glyceride is melted with a mixture of lopinavir, ritonavir, calcium silicate & crospovidone. Polysorbate 80 and water are added to the molten stearoyl macrogol glyceride. The wet mass is sized through a specified sieve and dried. The dried granules are sized through a specified sieve. This dried granular mass is blended with microcrystalline cellulose, crospovidone and lubricated with magnesium stearate.

According to another aspect of the present invention, there is provided a solid oral composition comprising one or more antiretroviral drugs and at least one water insoluble polymer, wherein the ratio of drug:polymer is 1:1 to 1:6. The most preferable antiretroviral drugs to be used are protease inhibitors such as lopinavir, ritonavir, amprenavir, saquinavir and others or their corresponding pharmaceutically acceptable salts, solvates, or hydrates. The protease inhibitors may also be the pharmaceutically acceptable enantiomers, derivatives, polymorphs or prodrugs thereof.

The or each water insoluble polymer that can be used, according to the present invention, may comprise acrylic copolymers e.g. Eudragit E100 or Eudragit EPO; Eudragit L30D-55, Eudragit FS30D, Eudragit RL30D, Eudragit RS30D, Eudragit NE30D, Acryl-Eze (Colorcon Co.); polyvinylacetate, for example, Kollicoat SR 3OD (BASF Co.); cellulose derivatives such as ethylcellulose, cellulose acetate e.g. Surelease (Colorcon Co.), Aquacoat ECD and Aquacoat CPD (FMC Co.). Most preferable being Eudragit E100 and is present in the range wherein the ratio of drug to polymer is 1:1 to 1:6.

The water soluble polymer that can be used, according to the present invention, may comprise homopolymers and co-polymers of N-vinyl lactams, especially homopolymers and co-polymers of N-vinyl pyrrolidone e.g. polyvinylpyrrolidone (PVP), co-polymers of PVP and vinyl acetate, co-polymers of N-vinyl pyrrolidone and vinyl acetate or vinyl propionate, cellulose esters and cellulose ethers, high molecular polyalkylene oxides such as polyethylene oxide and polypropylene oxide and co-polymers of ethylene oxide and propylene oxide. It is present in the range wherein the ratio of drug to polymer is 1:1 to 1:6.

As mentioned above, that the present invention can be manufactured through various techniques.

Accordingly, the present inventors have surprisingly found that when, by a process comprising hot melt extrusion of one or more drugs with one or more water insoluble polymers, optionally in combination with one or more water soluble polymers, the resulting product acquires taste masking property wherein the ratio of drug:polymer is 1:1 to 1:6.

It was surprisingly found that while carrying out the melt extrusion process an insitu reaction occurred between the drug and polymer. This insitu reaction led to ionic interaction between the drug and polymer eventually leading to taste masked product.

In general terms, the process of hot melt extrusion is carried out in the conventional extruders as known to a person skilled in the art.

The melt-extrusion process comprises the steps of preparing a homogeneous melt of one or more drugs, the polymer and the excipients, and cooling the melt until it solidifies. “Melting” means a transition into a liquid or rubbery state in which it is possible for one component to get embedded homogeneously in the other. Typically, one component will melt and the other components will dissolve in the melt thus forming a solution. Melting usually involves heating above the softening point of the polymer. The preparation of the melt can take place in a variety of ways. The mixing of the components can take place before, during or after the formation of the melt. For example, the components can be mixed first and then melted or be simultaneously mixed and melted. Usually, the melt is homogenized in order to disperse the active ingredients efficiently. Also, it may be convenient first to melt the polymer and then to mix in and homogenize the active ingredients.

Usually, the melt temperature is in the range of about 70° C. to about 200° C., preferably from about 80° C. to about 180° C., most preferred from about 90° C. to about 150° C.

Suitable extruders include single screw extruders, intermeshing screw extruders or else multiscrew extruders, preferably twin screw extruders, which can be co-rotating or counter-rotating and, optionally, be equipped with kneading disks. It will be appreciated that the working temperatures will also be determined by the kind of extruder or the kind of configuration within the extruder that is used.

The extrudates can be in the form of beads, granulates, tube, strand or cylinder and this can be further processed into any desired shape.

The term ‘extrudates’ as used herein refers to solid product solutions, solid dispersions and glass solutions of one or more drugs with one or more polymers and optionally pharmaceutically acceptable excipients.

According to another preferred embodiment, a powder blend of the one or more active drug(s) and polymers and optionally pharmaceutical excipients are transferred by a rotating screw of a single screw extruder through the heated barrel of an extruder whereby the powder blend melts and molten solution product is collected on a conveyor where it is allowed to cool to form an extrudate. Shaping of the extrudate can be conveniently be carried out by a calendar with two counter-rotating rollers with mutually matching depressions on their surface. A broad range of tablet forms can be attained, by using rollers with different forms of depressions. Alternatively, the extrudate is cut into pieces after solidification and can be further processed into suitable dosage forms. More preferably the extrudates thus finally obtained from the above process are then milled and ground to granules by the means known to a person skilled in the art.

Further, hot melt extrusion is a fast, continuous manufacturing process without requirement of further drying or discontinuous process steps; it provides short thermal exposure of active allows processing of heat sensitive actives; process temperatures can be reduced by addition of plasticizers; comparatively lower investment for equipment as against other processes. The entire process is anhydrous and the intense mixing and agitation of the powder blend that occur during processing contribute to a very homogenous extrudate(s).

In one aspect, the preferred embodiment in accordance with the present invention may comprise one or more antiretroviral drug(s) and one or more water insoluble polymers which are melt extruded by the process as described herein, where a powder blend of two antiretroviral drugs most preferably one or more protease inhibitor drugs i.e. lopinavir or its pharmaceutically acceptable salts, solvates or hydrates & ritonavir or its pharmaceutically acceptable salts, solvates, or hydrates, and a water insoluble polymer, or a combination of a water soluble and insoluble polymer, and other excipients which may comprise suitable bulking agents and flavourants. These are so processed to form a powder blend which is transferred through the heated barrel of the extruder most preferably single screw extruder, whereby the powder blend melts and molten solution product is collected on a conveyor whereby it is allowed to cool and form an extrudate. Alternatively, the extrudate is cut into pieces after solidification and can be further processed into suitable dosage forms. More preferably the extrudates thus finally obtained from the above process are then milled and ground to granules by the means known to a person skilled in the art.

In another aspect, the preferred embodiment in accordance with the present invention may comprise one or more antiretroviral drugs and a combination of one or more water insoluble polymers and one or more water soluble polymers which are melt extruded by the process as described herein, where a powder blend of two antiretroviral drugs, most preferably one or more protease inhibitor drugs i.e. lopinavir or its pharmaceutically acceptable salts, solvates or hydrates and ritonavir or its pharmaceutically acceptable salts, solvates or hydrates, and a combination of water soluble polymers & water insoluble polymers and other excipients which may comprise suitable bulking agents, plasticizer and flavourants.

These are so processed to form a powder blend which are transferred through the heated barrel of the extruder, whereby the powder blend melts and molten solution product is collected on a conveyor whereby it is allowed to cool and form an extrudate. Alternatively, the extrudate is cut into pieces after solidification and can be further processed into suitable dosage forms. More preferably the extrudates thus finally obtained from the above process are then milled and ground to granules by the means known to a person skilled in the art.

The water soluble polymers that can be used, according to the present invention, comprises of homopolymers and co-polymers of N-vinyl lactams, especially homopolymers and co-polymers of N-vinyl pyrrolidone e.g. polyvinylpyrrolidone (PVP), co-polymers of PVP and vinyl acetate, co-polymers of N-vinyl pyrrolidone and vinyl acetate or vinyl propionate, cellulose esters and cellulose ethers, high molecular polyalkylene oxides such as polyethylene oxide and polypropylene oxide and co-polymers of ethylene oxide and propylene oxide. It is present in the range wherein the ratio of drug to polymer is 1:1 to 1:6.

The water insoluble polymer that can be used, according to the present invention, comprises of acrylic copolymers e.g. Eudragit E100 or Eudragit EPO; Eudragit L30D-55, Eudragit FS30D, Eudragit RL30D, Eudragit RS30D, Eudragit NE30D, Acryl-Eze (Colorcon Co.); polyvinylacetate, for example, Kollicoat SR 3OD (BASF Co.); cellulose derivatives such as ethylcellulose, cellulose acetate e.g. Surelease (Colorcon Co.), Aquacoat ECD and Aquacoat CPD (FMC Co.). Most preferable being Eudragit E100 and is present in the range wherein the ratio of drug to polymer is 1:1 to 1:6.

Plasticizers can be incorporated depending on the polymer and the process requirement. These, advantageously, when used in the hot melt extrusion process decrease the glass transition temperature of the polymer. Plasticizers also help in reducing the viscosity of the polymer melt and thereby allow for lower processing temperature and extruder torque during hot melt extrusion. Examples of plasticizers which can be used in the present invention, include, but are not limited to, sorbitan monolaurate (Span 20), sorbitan monopalmitate, sorbitan monostearate, sorbitan monoisostearate; citrate ester type plasticizers like triethyl citrate, citrate phthalate; propylene glycol; glycerin; low molecular weight polyethylene glycol; triacetin; dibutyl sebacate, tributyl sebacate; dibutyltartrate, dibutyl phthalate. It may be present in an amount ranging from 0% to 10% to the weight of polymer.

According to a preferred embodiment, the present invention may be formulated for pediatric patients and from the point of view of pediatric patient acceptability suitable bulking agents that may be incorporated may comprise saccharides, including monosaccharides, disaccharides, polysaccharides and sugar alcohols such as arabinose, lactose, dextrose, sucrose, fructose, maltose, mannitol, erythritol, sorbitol, xylitol lactitol, and other bulking agents such as powdered cellulose, microcrystalline cellulose, purified sugar and derivatives thereof. Most preferably, purified sugar forms the bulking agent.

Accordingly, the present invention may further incorporate suitable pharmaceutically acceptable flavourants, for example citric acid, tartaric acid, lactic acid or other natural flavourants. The amount of flavourant ranges from about 0.5% to about 2% of the total weight of the water insoluble polymer.

It will be appreciated by those skilled in the art that the present invention may, if desired, be expanded to class of bitter drugs (i.e. pharmaceutical actives having an inherent bitter taste).

Examples of classes of drugs which may be used, include, but are not limited to, antiretrovirals such as protease inhibitors e.g. lopinavir, ritonavir, saquinavir, amprenavir, atazanavir, tipranavir, fosamprenavir and other class of drugs like reverse transcriptase inhibitors like lamividine, stavudine, zidovudine, emtricitabine, abacavir, adefovir, tenofovir; macrolide antibiotics such as erythromycin and clarithromycin, azithromycin which belongs to azalide class of macrolide antibiotics, penicillins such as cloxacillin sodium, amoxicillin and ampicillin, oxazolidinones such as linezolid, tricyclic antihistaminics such as desloratadine, cephalosporins such as cefuroxime, tetracyclic antibiotics such as chloramphenicol, fluoroquinolones such as ciprofloxacin, analgesics such as acetaminophen, acetyl salicylic acid and ibuprofen, decongestants such as phenylephrine hydrochloride and pseudoephedrine hydrochloride, antihistaminics such as chlorpheniramine and cetirizine, mucolytics such as ambroxol and bromhexine, anti-epileptic agents such as phenyloin and sodium valproate, Non steroidal anti-inflammatory drugs like indomethacin, ibuprofen, ketoprofen, fenoprofen; hormones like hydrocortisone, 17β estradiol hemihydrate; and other class of drugs including carbamazepine, theophylline, lidocaine and narcotic class of drugs. It will be appreciated that apart from the above drugs, their pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof can be used. Thus, according to a further aspect of the present invention, there is provided a solid oral composition comprising one or more of the above actives and the compositions may comprise the same excipients as described in relation to antiretroviral drugs. The compositions may be prepared in the same way as described in relation to antiretroviral drugs.

It will be further appreciated by a person skilled in the art, that melt extrusion with certain water insoluble polymers leads to an increase in solubility of poorly soluble drugs.

The following examples are for the purpose of illustration of the invention only and are not intended in any way to limit the scope of the present invention.

### Example 1

| Sr. | | Qty/Tab. |
|---|---|---|
| No | Ingredients | (mg) |
| I | DRY MIX | |
| 1. | Lopinavir | 200.00 |
| 2. | Ritonavir | 50.00 |
| 3. | Calcium silicate | 150.0 |
| 4. | Crospovidone | 50.0 |
| II | BINDER | |
| 5. | Stearoyl macrogol glyceride | 50.00 |
| 6. | Polysorbate | 20.0 |
| 7. | Purified water | q.s. |
| III | BLENDING | |
| 8. | Crospovidone | 75.0 |
| 9. | Calcium silicate | 50.0 |
| 10. | Avicel (microcrystalline cellulose) | 50.0 |
| IV. | LUBRICATION | |
| 11. | Magnesium Stearate | 5.00 |
| | Total | 700.0 |
| V | SEAL COATING | |
| 12. | HPMC | 4.0 |
| 13. | Talc | 1.0 |
| 14. | Methylene chloride | q.s. |
| 15. | Isopropyl alcohol | q.s. |
| VI. | AMB coating | |
| 16. | Ready colour mix system | 25.0 |
| 17. | Purified water | q.s. |
| | Total | 730.0 |

### Manufacturing Process—

Lopinavir, Ritonavir, Crospovidone Calcium silicate are mixed for 15 minutes and granulated by using Stearoyl macrogol glyceride, Polysorbate and purified water (melt granulation). Then Crospovidone, Calcium silicate and Avicel are blended with the dried granules and lubricated by using magnesium stearate. The lubricated granules are then compressed in to the tablets. Compressed tablets coated with seal coating solution are finally coated with an aqueous moisture barrier (AMB) film coat.

### Example 2

| Sr. | | Qty/Tab. |
|---|---|---|
| No. | Ingredients | (mg) |
| I. | DRY MIX | |
| 1. | Lopinavir | 200.00 |
| 2. | Ritonavir | 50.00 |
| 3. | Calcium silicate | 150.00 |
| 4. | Crospovidone | 50.0 |
| II. | BINDER | |
| 5. | Stearoyl macrogol glyceride | 50.0 |
| 6. | Polysorbate | 20.0 |
| 7. | Purified water | q.s. |
| III. | BLENDING | |
| 8. | Crospovidone | 50.0 |
| 9. | Microcrystalline Cellulose | 70.0 |
| IV. | LUBRICATION | |
| 10. | Magnesium Stearate | 10.0 |
| | Total | 650.0 |
| V. | FILM COATING | |
| 11. | Ready colour mix system | 12.0 |
| 12. | Purified water | — |
| | Total | 662.0 |

### Manufacturing Process—

Lopinavir, Ritonavir, Crospovidone, Calcium silicate are mixed for 15 minutes and granulated by using Stearoyl macrogol glyceride, Polysorbate and purified water (melt granulation). Then Crospovidone and avicel are blended with the dried granules and lubricated by using magnesium stearate. The lubricated granules are then compressed in to tablets. Compressed tablets are finally coated with film coat.

### Example 3

| Sr. | | Qty/Tab. |
|---|---|---|
| No. | Ingredients | (mg) |
| I. | DRY MIX | |
| 1. | Lopinavir | 200.00 |
| 2. | Ritonavir | 50.00 |
| 3. | Calcium silicate | 150.00 |
| 4. | Crospovidone | 50.0 |
| II. | BINDER | |
| 5. | Stearoyl macrogol glyceride | 50.0 |
| 6. | Purified water | q.s. |
| III. | BLENDING | |
| 7. | Crospovidone | 50.0 |
| 8. | Microcrystalline Cellulose | 90.0 |
| IV. | LUBRICATION | |
| 9. | Magnesium Stearate | 10.0 |
| | Total | 650.0 |
| V. | FILM COATING | |
| 10. | Ready colour mix system | 12.0 |
| 11. | Purified water | — |
| | Total | 662.0 |

### Manufacturing Process—

Lopinavir, Ritonavir, Crospovidone, Calcium silicate are mixed for 15 minutes and granulated by using Stearoyl macrogol glyceride in purified water (melt granulation). Then Crospovidone and avicel are blended with the dried granules and lubricated by using magnesium stearate. The lubricated granules are then compressed into tablets. Compressed tablets are finally coated with film coat

### Example 4

| Sr. | | Qty/Tab. |
|---|---|---|
| No. | Ingredients | (mg) |
| I. | DRY MIX | |
| 1. | Lopinavir | 200.00 |
| 2. | Ritonavir | 50.00 |
| 3. | Calcium silicate | 150.00 |
| 4. | Crospovidone | 50.0 |
| II. | BINDER | |
| 5. | Polysorbate | 20.0 |
| 6. | Purified water | q.s. |
| III. | BLENDING | |
| 7. | Crospovidone | 50.0 |
| 8. | Microcrystalline Cellulose | 120 |
| IV. | LUBRICATION | |
| 9. | Magnesium Stearate | 10.0 |
| | Total | 650.0 |
| V. | FILM COATING | |
| 10. | Ready colour mix system | 12.0 |
| 11. | Purified water | — |
| | Total | 662.0 |

### Manufacturing Process—

Lopinavir, Ritonavir, Crospovidone and Calcium silicate are mixed for 15 minutes and granulated by using Polysorbate and purified water. Then Crospovidone and avicel are blended with the dried granules and lubricated by using magnesium stearate. The lubricated granules are then compressed into tablets. Compressed tablets are finally coated with film coat.

### Example 5

| Sr. | | Qty/Tab. |
|---|---|---|
| No. | Ingredients | (mg) |
| I. | DRY MIX | |
| 1. | Lopinavir | 200.00 |
| 2. | Ritonavir | 50.00 |
| 3. | Calcium silicate | 150.00 |
| 4. | Crospovidone | 50.0 |
| II. | BINDER | |
| 5. | Stearoyl macrogol glyceride | 50.0 |
| 6. | Chremophore | 20.0 |
| 7. | Purified water | q.s. |
| III. | BLENDING | |
| 8. | Crospovidone | 50.0 |
| 9. | Microcrystalline Cellulose | 70.0 |
| IV. | LUBRICATION | |
| 10. | Magnesium Stearate | 10.0 |
| | Total | 650.0 |
| V. | FILM COATING | |
| 11 | Ready colour mix system | 12.0 |
| 12. | Purified water | — |
| | Total | 662.0 |

### Manufacturing Process—

Lopinavir, Ritonavir, Crospovidone and Calcium silicate are mixed for 15 minutes and granulated by using Stearoyl macrogol glyceride, Chremophore and purified water (melt granulation). Then Crospovidone and avicel are blended with the dried granules and lubricated by using magnesium stearate. The lubricated granules are then compressed into tablets. Compressed tablets are finally coated with film coat.

### Example 6

| Sr. | | Qty/Tab. |
|---|---|---|
| No. | Ingredients | (mg) |
| I. | DRY MIX | |
| 1. | Lopinavir | 200.00 |
| 2. | Ritonavir | 50.00 |
| 3. | Calcium silicate | 150.00 |
| 4. | Crospovidone | 50.0 |
| II. | BINDER | |
| 5. | Chremophore | 20.0 |
| 6. | Purified water | q.s. |
| III. | BLENDING | |
| 7. | Crospovidone | 50.0 |
| 8. | Microcrystalline Cellulose | 120 |
| IV. | LUBRICATION | |
| 9. | Magnesium Stearate | 10.0 |
| | Total | 650.0 |
| V. | FILM COATING | |
| 10. | Ready colour mix system | 12.0 |
| 11. | Purified water | — |
| | Total | 662.0 |

### Manufacturing Process—

Lopinavir, Ritonavir, Crospovidone and Calcium silicate are mixed for 15 minutes and granulated by using Chremophore and purified water. Then Crospovidone and avicel are blended with the dried granules and lubricated by using magnesium stearate. The lubricated granules are then compressed into tablets. Compressed tablets are finally coated with film coat.

### Example 7

| Sr. | | Qty/Tab. |
|---|---|---|
| No. | Ingredients | (mg) |
| I. | DRY MIX | |
| 1. | Lopinavir | 200.00 |
| 2. | Ritonavir | 50.00 |
| 3. | Calcium silicate | 150.00 |
| 4. | Crospovidone | 50.0 |
| II. | BINDER | |
| 5. | Chremophore | 10.0 |
| 6. | Span | 10.0 |
| 7. | Purified water | q.s. |
| III. | BLENDING | |
| 8. | Crospovidone | 50.0 |
| 9. | Microcrystalline Cellulose | 120 |
| IV. | LUBRICATION | |
| 10. | Magnesium Stearate | 10.0 |
| | Total | 650.0 |
| V. | FILM COATING | |
| 11. | Ready colour mix system | 12.0 |
| 12. | Purified water | — |
| | Total | 662.0 |

### Manufacturing Process—

Lopinavir, Ritonavir, Crospovidone and Calcium silicate are mixed for 15 minutes and granulated by using Span, Chremophore and purified water. Then Crospovidone and avicel are blended with the dried granules and lubricated by using magnesium stearate. The lubricated granules are then compressed into tablets. Compressed tablets are finally coated with film coat.

### Example 8

| | | Qty/ |
|---|---|---|
| Sr. | | tablet |
| No. | Ingredients | (mg) |
| I | ACTIVE PART | |
| 1. | Lopinavir | 200.0 |
| 2. | Ritonavir | 50.0 |
| 3. | Colloidal Silicon Dioxide | 10.0 |
| II | BINDER | |
| 4. | Copovidone (Kollidon VA64) | 800.0 |
| 5. | Sorbitan Monolaurate (Span 20) | 80.0 |
| III | EXTRAGRANULAR | |
| 6. | Microcrystalline Cellulose (Avicel PH 102) | 124.0 |
| 7. | Crospovidone (Kollidon) | 110.0 |
| 8. | Colloidal Silicon Dioxide | 18.0 |
| IV | LUBRICATION | |
| 9. | Sodium Stearyl Fumarate | 8.0 |
| | Total | 1400.0 |
| V. | SEAL COATING | |
| 10 | Hypromellose (6 cps) | 5.00 |
| 11 | Isopropyl alcohol | q.s. |
| 12 | Purified Water | q.s. |
| VI | FILM COATING | |
| 13. | Ready Colour Mix System | 15.0 |
| 14. | Purified Water | q.s. |
| | Total | 1420.0 |

### Manufacturing Process

Lopinavir, Ritonavir, Colloidal silicon dioxide are mixed for 15 minutes and granulated by using copovidone and sorbitan monolaurate (melt granulation). Then, microcrystalline cellulose, crospovidone and colloidal silicon dioxide are blended with the dried granules and lubricated by using sodium stearyl fumarate. The lubricated granules are then compressed into tablets. Compressed tablets coated with seal coating solution are finally film coated with ready colour mix system.

### Example 9

| Sr. | | QTY |
|---|---|---|
| No. | INGREDIENTS | (mg/Sachet) |
| 1. | Lopinavir | 200.0 |
| 2. | Ritonavir | 50.0 |
| 3. | Kollidon VA 64 | 400.0 |
| | (PVP:vinyl acetate) | |
| 4. | Eudragit E100 | 400.0 |
| 5. | Span 20 | 40.0 |
| 6. | Pharma grade Sugar | 894.0 |
| 7. | Flavor | 16.0 |
| | Total | 2000.0 |

### Manufacturing Process:—

The actives Lopinavir & Ritonavir along with Eudragit E100 were sieved, sifted & mixed together in a mixer. Kollidon VA 64 (6:4) was mixed separately with Span 20 in a granulator and the mixture was then sifted through 8# or 12#. This mixture was then further finally mixed with the above portion of the actives and Eudragit E100. The whole mixture was then extruded in a hot melt extruder. The melting temperature for the extrusion process ranges from 70 to 200° C. Most preferably at a temperature range being carried out at 90 to 150° C. After adding above all ingredients, pharma grade sugar and suitable flavor was added in the extruder. After the process, the molten mass thus obtained are collected on a conveyor where they are cooled to form extrudates and these extrudates on further processing are converted into granules and filled in sachets.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

1. A solid oral composition comprising at least two protease inhibitors or their pharmaceutically acceptable salts, solvates, hydrates, enantiomers, derivatives, polymorphs or prodrugs and at least one water insoluble polymer, wherein the ratio of drug to polymer in the composition ranges from about 1:1 to about 1:6.

2-4. (canceled)

5. The solid oral composition according to claim 1, wherein the or each protease inhibitor is selected from lopinavir, ritonavir, amprenavir, saquinavir or their pharmaceutically acceptable salts, solvates, hydrates, enantiomers, derivatives, polymorphs or prodrugs.

6. The solid oral composition according to claim 1, wherein the two protease inhibitors are lopinavir and ritonavir or their pharmaceutically acceptable salts, solvates, hydrates, enantiomers, derivatives, polymorphs or prodrugs.

7. (canceled)

8. The solid oral composition according to claim 1, wherein the or each water insoluble polymer is selected from the group consisting of acrylic copolymers; polyvinylacetate; cellulose derivatives; and cellulose acetates.

9. The solid oral composition according to claim 1, wherein the or each water insoluble polymer is selected from Eudragit E100, Eudragit EPO, Eudragit L30D-55, Eudragit FS30D, Eudragit RL30D, Eudragit RS30D, Eudragit NE30D, Acryl-Eze, Kollicoat SR 3OD, ethylcellulose, Surelease, Aquacoat ECD and Aquacoat CPD.

10-13. (canceled)

14. The solid oral composition according to claim 1, wherein the composition further comprises at least one water soluble polymer.

15. The solid oral composition according to claim 14, wherein the water soluble polymer is selected from the group consisting of homopolymers and co-polymers of N-vinyl lactams, a homopolymer or co-polymers of polyvinylpyrrolidone and vinyl acetate, co-polymers of N-vinyl pyrrolidone and vinyl acetate or vinyl propionate; high molecular polyalkylene oxides and co-polymers of ethylene oxide and propylene oxide.

16. The solid oral composition according to claim 15, wherein the water soluble polymer is selected from the group consisting of polyvinylpyrrolidone, Kollidon VA 64, polyethylene oxide and polypropylene oxide.

17-19. (canceled)

20. The solid oral composition according to claim 1, wherein the composition further comprises a plasticizer.

21. The solid oral composition according to claim 20, wherein the plasticizer is selected from the group consisting of a polysorbate, a citrate ester, propylene glycol, glycerin, low molecular weight polyethylene glycol, triacetin, dibutyl sebacate, tributyl sebacate, dibutyltartrate and dibutyl phthalate.

22. The solid oral composition according to claim 21, wherein the plasticizer is selected from the group consisting of sorbitan monolaurate, sorbitan monopalmitate, sorbitan monostearate, sorbitan monoisostearate, triethyl citrate and citrate phthalate.

23. (canceled)

24. The solid oral composition according to claim 20, wherein the plasticizer is present in an amount of up to about 10% of the weight of polymer.

25-31. (canceled)

32. A process for preparing a solid oral composition according to claim 1 comprising the steps: (a) preparing a homogeneous melt of the or each protease inhibitor; the or each water insoluble polymer and the or each excipients; (b) cooling the melt obtained in step (a); (c) allowing the cooled melt to solidify to obtain extrudates; and (d) processing the extrudates into a desired shape.

33-41. (canceled)

42. A process for preparing a solid oral composition according to claim 1 comprising: (a) melt granulating one or more solubility enhancers and one or more first pharmaceutically acceptable excipients with the or each protease inhibitor in purified water to form a granulated material; (b) sieving the granulated material; (c) drying the sieved granulated material to form dried granules; (d) lubricating the dried granules with one or more lubricants and one or more second pharmaceutically acceptable excipients; and (e) optionally further processing the lubricated dried granules.

43-55. (canceled)

56. The process according to claim 42, wherein the or each solubility enhancers are selected from the group consisting of stearoyl macrogol glyceride, a polysorbate, and polyoxyl castor oil.

57-58. (canceled)

59. The process according to claim 42, wherein the first pharmaceutically acceptable excipients and second pharmaceutically acceptable excipients independently of one another are selected from the group consisting of polymers, fillers or diluents, surfactants, solubility enhancers, disintegrants, binders, lubricants, non-ionic solubilisers, glidants and combinations thereof.

60-61. (canceled)

62. The process according to claim 42, wherein the first pharmaceutically acceptable excipients and second pharmaceutically acceptable excipients independently of one another comprise one or more diluents and one or more disintegrants.

63-76. (canceled)

77. A method comprising utilizing the composition according to claim 1 in medicine.

78. A method comprising utilizing the composition according to claim 1 in the manufacture of a medicament for treating HIV.

79. A method of treating HIV comprising administering to a patient a therapeutically effective amount of a composition according to claim 1.

80-81. (canceled)
